(19) United States
(12) Patent Application Publication
(10) Pub. No.: US 2012/0107397 A1
(43) Pub. Date: May 3, 2012

(54) PHARMACEUTICAL COMPOSITIONS OF VALSARTAN

(75) Inventors: Bandi Parthasaradhi Reddy, Hyderabad (IN); Male Srinivas Reddy, Hyderabad (IN); Pothireddy Venkateswar Reddy, Hyderabad (IN); Muppidi Vanaja Kumari, Hyderabad (IN)

(73) Assignee: Hetero Research Foundation, Hyderabad (IN)

(21) Appl. No.: 13/381,963

(22) PCT Filed: Jul. 3, 2009
(86) PCT No.: PCT/IN2009/000376

§ 371 Date: Jan. 2, 2012

(51) Int. Cl. A61K 31/41; A61P 9/12; A61K 9/20; A61K 31/549; A61K 31/4422

(52) U.S. Cl. 424/464; 514/381; 514/223.5; 514/356

(57) ABSTRACT

The present invention relates to the stable pharmaceutical composition comprising valsartan or a pharmaceutically acceptable salt thereof with mannitol as a filler and povidone as binder. The present invention also relate to the valsartan or pharmaceutically acceptable salt thereof in combination with hydrochlorothiazide or amlodipine or both; and optionally one or more additional excipients.

### FIELD OF THE INVENTION

The present invention relates to the stable pharmaceutical composition comprising valsartan or a pharmaceutically acceptable salt thereof with mannitol as a filler and povidone as binder. The present invention also relate to the valsartan or pharmaceutically acceptable salt thereof in combination with hydrochlorothiazide or amlodipine or both; and optionally one or more additional excipients.

### BACKGROUND OF THE INVENTION

Valsartan, a compound having the chemical name N-(1-oxopentyl)-N-[[2′-(1H-tetrazol-5-yl) [1,1′-biphenyl]-4-yl]methyl]-L-valine.

Valsartan was first disclosed in the U.S. Pat. No. 5,399,578, which is incorporated by reference. It is an angiotensin II antagonist, known to be effective in the treatment of congestive heart failure and reducing blood pressure irrespective of age, sex or race and is also well tolerated.

Valsartan is commercially available as 40 mg, 80 mg, 160 mg, 320 mg tablets. It is sold under the name Diovan.

Valsartan and hydrochlorothiazide is commercially available as 80 mg/12.5 mg, 160 mg/12.5 mg, 160 mg/25 mg, 320 mg/12.5 mg, 320 mg/25 mg combination tablets. It is sold under the name Diovan HCT®.

Valsartan and amlodipine is commercially available as 160 mg/5 mg, 160 mg/10 mg, 320 mg/5 mg, 320 mg/10 mg combination tablets. It is sold under the name EXFORGE.

Amlodipine, valsartan and hydrochlorothiazide is commercially available as 5/160/12.5 mg, 10/160/12.5 mg, 5/160/25 mg, 10/160/25 mg and 10/320/25 mg combination tablets. It is sold under the name Exforge HCT.

U.S. Pat. Nos. 6,294,197; 6,485,745; 6,858,228 and EP patent No. 1,410,797, which is incorporated by reference, describes a compressed solid oral dosage from comprising of valsartan and hydrochlorothiazide, where the active constituent is more than 35% by weight based on total weight of the compressed solid oral dosage form, of the active ingredient, obtained by dry granulation.

WO Patent Application Publication No. 2005/041941 relates to a pharmaceutical composition containing valsartan, optionally a combination of valsartan with hydrochlorothiazide, obtainable by direct tabletting.

WO Patent Application Publication No. 2005/082329 relates to a solid dosage form comprising a core contains valsartan and a coating layer contains hydrochlorothiazide.

WO Patent Application Publication No. 96/31234 describes a pharmaceutical combination composition comprising benazepril or benazeprilat and valsartan.

U.S. Pat. No. 6,395,728, which is incorporated by reference, describes method for the treatment of hypertension, effective amount of valsartan and amlodipine.

EP Patent Application No. 1,994,926 describes a pharmaceutical formulation in the form of a tablet consisting of 20% to 34% of valsartan, microcrystalline cellulose and pregelatinized starch in a weight ratio between 1:1 and 5:1, colloidal silicon dioxide and magnesium stearate, the tablets prepared by direct compression.

WO Patent Application Publication No. 2006/113631 relates to a composition comprising valsartan and a solubility enhancing agent.

WO Patent Application Publication No. 2008/076780 relates to a composition comprising a solid dispersion of amorphous valsartan and a solubility-enhancing polymer.

EP Patent No. 1,682,122 describes a combination of valsartan, amiloride or triameterine and diuretic.

EP Patent No. 1,507,529, which is incorporated by reference, a combination of valsartan, amlodipide and hydrochlorothiazide.

U.S. patent application No. 2003/0152620 describes an oral solid pharmaceutical composition comprising pharmacologically effective amounts of valsartan and which is, on average, at least 1.2 times more bioavailabe than a valsartan capsules.

WO Patent Application Publication No. 97/49394 discloses compressed solid oral dosage forms, e.g. by compaction, of valsartan, optionally in salt form, optionally combined with hydrochlorothiazide.

All the above mentioned patents are incorporated by references.

Inspite of all the above disclosed formulations of valsartan, still there is a need for oral solid pharmaceutical compositions that are having good dissolution, good stability and robust process.

Thus an object of the present invention is to provide the pharmaceutical composition of valsartan, having good dissolution, good stability and robust pharmaceutical composition, which enable to have a formulation without any difficulty.

Another object of the present invention provides a process for preparing of valsartan or pharmaceutically acceptable salt thereof.

Another object of the present invention is to provide the pharmaceutical composition of valsartan or pharmaceutically acceptable salt thereof in combination with hydrochlorothiazide or amlodipine; or both, having good dissolution, good stability and robust pharmaceutical composition, which enable to have a formulation without any difficulty.

Another object of the present invention provides a process for preparing of valsartan or pharmaceutically acceptable salt thereof in combination with hydrochlorothiazide or amlodipine; or both.

### DETAILED DESCRIPTION OF THE INVENTION

According to one aspect of the present invention, there is provided the pharmaceutical composition comprising valsartan or a pharmaceutically acceptable salt thereof with mannitol as a filler and povidone as binder, and optionally one or more additional excipients.

The pharmaceutical composition of the present invention has been found to have reliable and robust oral formulation in comparison with literature oral solid dosage forms, for example, the oral dosage from reported in U.S. Pat. No. 5,399,578.

It has been found that the pharmaceutical composition of the present invention not only display the stable and robust formulation, but also display the improved characteristics associated even with dissolution.

The additional excipients may be a filler agent, a disintegrant agent, a binder, a glidant and a lubricant.

Preferably, the pharmaceutical composition is oral solid dosage forms.

The pharmaceutical composition may be for example, in the form of a tablet, a caplet, pellets, a capsule, granules, a pill, powder or a sachet.

Preferably the pharmaceutical composition is in the form of a tablet and a capsule. The capsule may contain powder, compressed powder or granules.

Still more preferably, the oral solid pharmaceutical composition is in the form of a tablet.

In one embodiment of the present invention, the concentration of valsartan or a pharmaceutically acceptable salt thereof to mannitol in the pharmaceutical composition is about 1:0.25 to about 1:5.

More preferably of the present invention, the concentration of valsartan or a pharmaceutically acceptable salt thereof to mannitol in the pharmaceutical composition is about 1:0.5 to about 1:2.5.

In another embodiment of the present invention, the concentration of valsartan or a pharmaceutically acceptable salt thereof to povidone in the pharmaceutical composition is about 1:0.01 to about 1:0.5.

More preferably of the present invention, the concentration of valsartan or a pharmaceutically acceptable salt thereof to povidone in the pharmaceutical composition is about 1:0.02 to about 1:0.1.

In another embodiment of the present invention, the concentration of povidone to mannitol in the pharmaceutical composition is about 1:30 to about 1:90.

More preferably of the present invention, the concentration of povidone to mannitol in the pharmaceutical composition is about 1:50 to about 1:75.

In another embodiment, the pharmaceutical composition of the present invention comprises a filler agent (in addition to mannitol), a binder (in addition to povidone), a disintegrant agent, and a lubricant, wherein said solid pharmaceutical composition comprising valsartan or pharmaceutically acceptable salt thereof concentration below 35% of the total weight of the pharmaceutical composition.

More preferably valsartan or pharmaceutically acceptable salt thereof is concentration from about 20 to about 34% of the total weight of the pharmaceutical composition.

Excipient selection depends on various factors, such as, the choice of active ingredient, percentage, the objectives of the tablet formulation development and method of manufacture. The foremost property an excipient must possess is compatibility with active ingredients.

Preferably, the pharmaceutically acceptable excipients in accordance with the invention include at least one filler agent (in addition to mannitol), and/or at least one binder (in addition to povidone), and/or at least one disintegrant agent and/or at least one lubricant.

Preferably, the filler includes mannitol, microcrystalline cellulose, calcium carbonate, dibasic calcium phosphate, lactose, magnesium carbonate, magnesium oxide, lactose anhydrous, isomalt, sorbitol or mixtures thereof and more preferably mannitol and/or microcrystalline cellulose.

Preferably, the lubricants includes magnesium stearate, calcium to stearate, stearic acid, a salt of stearic acid, talc, sodium stearyl fumarate, glyceryl behenate, magnesium silicate, magnesium trisilicate, macrogol, talc, hydrogenated castor oil or mixtures thereof and more preferably magnesium stearate and/or calcium stearate.

Preferably, the disintegratrants includes croscarmellose sodium, starch, sodium starch glycolate, crospovidone, carboxymethyl cellulose calcium, carboxymethylcellulose sodium, magnesium aluminium silicate or mixtures thereof and more preferably croscarmellose sodium and/or starch.

Preferably, the glidant may be for example colloidal anhydrous silica, talc or mixtures thereof.

Preferably, the binder includes polyvinylpyrrolidone k-30, hydroxypropyl cellulose, hydroxypropyl methyl cellulose, hydroxypropyl cellulose (low-substituted), starch or mixtures thereof and more preferably polyvinylpyrrolidone k-30 and/or hydroxypropyl cellulose.

Preferably,, the coating agent includes hydroxypropyl methyl cellulose, polyvinyl alcohols, hydroxypropyl cellulose, macrogols or mixtures thereof and more preferably hydroxypropyl methyl cellulose and/or polyvinyl alcohols.

Preferably, the coloring agent includes titanium dioxide, red iron oxide, yellow iron oxide, black iron oxides and mixture thereof.

Other ingredients such as stabilizers, antioxidants, and antiadherants, conventionally used for pharmaceutical formulations may also be included in the present formulation.

According to another aspect of the present, invention, there is provided the process for preparing the pharmaceutical composition, which comprises mixing valsartan or a pharmaceutically acceptable salt thereof, mannitol and povidone, and optionally one or more additional excipients.

It has been found that the pharmaceutical process of the present invention are physically stable to pharmaceutical unit operations such as compression, thereby making oral solid dosage of the present invention amenable to pharmaceutical compounding operations, such as for example, tabletting.

The additional excipients may be a filler agent, a disintegrant agent, a binder, a glidant and a lubricant.

Preferably, the oral solid pharmaceutical composition prepared according to the process of the invention may be for example, in the form of a tablet, a caplet, a pellet, a capsule, granules, a pill, powder or a sachet.

Still more preferably, the oral solid pharmaceutical composition is in the form of a tablet.

Preferably, the tablet compositions are prepared by process of direct compression, wet granulation or slugging or roll compaction, more preferably wet granulation.

The tablet may be also optionally coated with a coating agent.

The preferred embodiment of the invention is suitable for forming valsartan tablet comprising in parts by weight from about 20% to about 35% valsartan or a pharmaceutically acceptable salt thereof, from about 20% to about 80% mannitol and/or lactose, from about 0.25% to about 10% povidone, from about 1% to about 15% starch, from about 0.5% to about 10% croscarmellose sodium or dibasic calcium phosphate or microcrystalline cellulose, from about 0.25% to about 5% magnesium stearate, from about 1.5% to about 4% opadry. Optionally additional excipient/s may be used. The additional excipients include pharmaceutical lubricants, disintegrators, binders, glidents, fillers or mixtures thereof.

The pharmaceutical composition of the present invention may be used for the treatment of hypertension.

According to another aspect of the present invention, there is provided the pharmaceutical composition comprising valsartan or a pharmaceutically acceptable salt thereof with mannitol as a filler, povidone as binder in combination with hydrochlorothiazide or amlodipine or both; and optionally one or more additional excipients.

The preferred embodiment of the present invention provides stable pharmaceutical formulations of combination products of i) valsartan and hydrochlorothiazide; ii) valsartan and amlodipine; or iii) valsartan, hydrochlorothiazide and amlodipine.

It has been found that the pharmaceutical composition of the present invention not only display the stable and robust formulation, but also display the improved characteristics associated even with dissolution.

The additional excipients may be a filler agent, a disintegrant agent, a binder, a glidant and a lubricant.

Preferably, the pharmaceutical composition is oral solid dosage forms.

The pharmaceutical composition may be for example, in the form of a tablet, a caplet, pellets, a capsule, granules, a pill, powder or a sachet.

Preferably the pharmaceutical composition is in the form of a tablet and a capsule. The capsule may contain powder, compressed powder or granules.

Still more preferably, the oral solid pharmaceutical composition is in the form of a tablet.

In another embodiment, the pharmaceutical composition of the present invention comprises a filler agent (in addition to mannitol), a binder (in addition to povidone), a disintegrant agent, and a lubricant, wherein said solid pharmaceutical composition comprising valsartan or a pharmaceutically acceptable salt thereof concentration below 35% with mannitol as a filler, povidone as binder in combination with hydrochlorothiazide and/or amlodipine of the total weight of the pharmaceutical composition and optionally one or more additional excipients.

More preferably valsartan or a pharmaceutically acceptable salt thereof concentration from about 20 to about 34% of the total weight of the pharmaceutical composition.

Excipient selection depends on various factors, such as, the choice of active ingredient percentage, the objectives of the tablet formulation development and method of manufacture. The foremost property an excipient must possess is compatibility with active ingredients.

Preferably, the pharmaceutically acceptable excipients in accordance with the invention include at least one filler agent (in addition to mannitol), and/or at least one binder (in addition to povidone), and/or at least one disintegrant agent and/or at least one lubricant.

Preferably, the filler includes mannitol, microcrystalline cellulose, calcium carbonate, dibasic calcium phosphate, lactose, magnesium carbonate, magnesium oxide, lactose anhydrous, isomalt, sorbitol or mixtures thereof and more preferably mannitol and/or microcrystalline cellulose.

Preferably, the lubricants includes magnesium stearate, calcium stearate, stearic acid, a salt of stearic acid, talc, sodium stearyl fumarate, glyceryl behenate, magnesium silicate, magnesium trisilicate, macrogol, talc, hydrogenated castor oil or mixtures thereof and more preferably magnesium stearate and/or calcium stearate.

Preferably, the disintegratrants includes croscarmellose sodium, starch, sodium starch glycolate, crospovidone, carboxymethyl cellulose calcium, to carboxymethylcellulose sodium, magnesium aluminium silicate or mixtures thereof and more preferably croscarmellose sodium and/or starch.

Preferably, the glidant may be for example colloidal anhydrous silica, talc or mixtures thereof.

Preferably, the binder includes polyvinylpyrrolidone k-30, hydroxypropyl cellulose, hydroxypropyl methyl cellulose, hydroxypropyl cellulose (low-substituted), starch or mixtures thereof and more preferably polyvinylpyrrolidone k-30 and/or hydroxypropyl cellulose.

Preferably, the coating agent includes hydroxypropyl methyl cellulose, polyvinyl alcohols, hydroxypropyl cellulose, macrogols or mixtures thereof and more preferably hydroxypropyl methyl cellulose and/or polyvinyl alcohols.

Preferably, the coloring agent includes titanium dioxide, red iron oxide, yellow iron oxide, black iron oxides and mixture thereof.

Other ingredients such as stabilizers, antioxidants, and antiadherants, conventionally used for pharmaceutical formulations may also be included in the present formulation.

According to another aspect of the present invention, there is provided the process for preparing the pharmaceutical composition, which comprises mixing valsartan or a pharmaceutically acceptable salt thereof in combination with hydrochlorothiazide or amlodipine or both; mannitol and povidone, and optionally one or more additional excipients.

It has been found that the pharmaceutical process of the present invention are physically stable to pharmaceutical unit operations such as compression, thereby making oral solid dosage of the present invention amenable to pharmaceutical compounding operations, such as for example, tabletting.

The additional excipients may be a filler agent, a disintegrant agent, a binder, a glidant and a lubricant.

Preferably, the oral solid pharmaceutical composition may be for example, in the form of a tablet, a caplet, pellets, a capsule, granules, a pill, powder or a sachet.

Still more preferably, the oral solid pharmaceutical composition is in the form of a tablet.

Preferably, the tablet composition are prepared by the process of direct compression, wet granulation or slugging or roll compaction, more preferably wet granulation.

The tablet may be also optionally coated with a coating agent.

The preferred embodiment of the invention is suitable for forming valsartan and hydrochlorothiazide tablet comprising in parts by weight from about 20% to about 35% valsartan or a pharmaceutically acceptable salt thereof, from about 0.2% to about 10% hydrochlorothiazide, from about 20% to about 80% mannitol, from about 0.25% to about 10% povidone, from about 1% to about 15% starch, from about 0.5% to about 10% croscarmellose sodium, from about 0.25% to about 5% magnesium stearate, from about 1.5% to about 4% opadry. Optionally additional excipient/s may be used. The additional excipients include pharmaceutical lubricants, disintegrators, binders, glidents, fillers or mixtures thereof.

The preferred embodiment of the invention is suitable for forming valsartan and amlodipine tablet comprising in parts by weight from about 20% to about 35% valsartan or a pharmaceutically acceptable salt thereof, from about 0.25% to about 10% amlodipine or a pharmaceutically acceptable salt thereof, from about 20% to about 80% mannitol, from about 0.25% to about 10% povidone, from about 1% to about 15% starch, from about 0.5% to about 10% croscarmellose sodium, from about 0.25% to about 5% magnesium stearate, from about 1.5% to about 4% opadry. Optionally additional excipient/s may be used. The additional excipients include pharmaceutical lubricants, disintegrators, binders, glidents, fillers or mixtures thereof.

The preferred embodiment of the invention is suitable for forming valsartan and amlodipine tablet comprising in parts by weight from about 20% to about 35% valsartan or a pharmaceutically acceptable salt thereof, from about 0.25% to about 10% amlodipine or a pharmaceutically acceptable salt thereof, from about 1% to about 10% hydrochlorothiazide, from about 20% to about 80% mannitol, from about 0.25% to about 10% povidone, from about 1% to about 15% starch, from about 1% to about 10% croscarmellose sodium, from about 0.25% to about 5% magnesium stearate, from about 1.5% to about 4% opadry. Optionally additional excipient/s may be used. The additional excipients include pharmaceutical lubricants, disintegrators, binders, glidents, fillers or mixtures thereof.

The pharmaceutical composition of the present invention may be used for the treatment of hypertension.

### EXAMPLES

### Example 1

Preparation of a valsartan tablet wherein the valsartan is present in the form of valsartan with mannitol and povidone:

This example demonstrates a tablet composition comprising valsartan as an active pharmaceutical ingredient and one or more pharmaceutically acceptable excipients, wherein said valsartan is present in the form of the valsartan with mannitol and povidone, in accordance with an embodiment of the invention. This example further demonstrates a process for preparing a solid pharmaceutical composition in accordance with an embodiment of the invention.

The tablets were prepared using the materials listed in table.

| Component | Weight (mg)/Tablet | | | | % (w/w) |
|---|---|---|---|---|---|
| Valsartan | 40 | 80 | 160 | 320 | 32 |
| Mannitol | 77.25 | 154.5 | 309 | 618 | 61.8 |
| Povidone | 1.25 | 2.5 | 5 | 10 | 1.0 |
| Purified water | q.s | q.s | q.s | q.s | — |
| Maize starch | 2.5 | 5 | 10 | 20 | 2 |
| Croscarmellose sodium | 2.5 | 5 | 10 | 20 | 2 |
| Magnesium stearate | 1.5 | 3 | 6 | 12 | 1.2 |
| Tablet weight | 125 | 250 | 500 | 1000 | — |
| Opadry | 2.5 | 5 | 10 | 20 | 2 |

The tablets were manufactured using the procedure comprising the following steps: valsartan and mannitol was done in a rapid mixer granulator and mixed for 5 to 20 minutes. Binder solution (povidone K-29/32 in purified water) is added to contents of rapid mixer granulator and mixed to get dough mass. The dough mass was dried, and dried granules passed through #18.

The blending of ingredients including above dried granules, maize starch, croscarmellose sodium and magnesium stearate were weighted, transferred into a blender, and mixed to form a homogeneous power mixture; Jo the resultant mixture was compressed into tablets of appropriate weight and hardness to obtain a valsartan tablet; and the tablets was coated with opadry.

The results of dissolution of valsartan tablets 320 mg is shown below.

| | | Valsartan |
|---|---|---|
| Time | Diovan | tablets |
| (minutes) | 320 mg | 320 mg |
| 10 | 95 | 98 |
| 20 | 97 | 99 |
| 30 | 97 | 99 |
| 45 | 97 | 100 |

The example demonstrates the dissolution properties of the tablet prepared in accordance with the invention.

The tablets of valsartan and commercially available valsartan tablets (i.e., Diovan) were tested for in vitro drug release in 1000 ml of 0.067 M phosphate buffer, pH 6.8 using a USP-2 apparatus speed operating at 50 rpm. The valsartan tablets 320 mg were greater than 85% in 10 minutes.

### Valsartan Tablets Stability Studies:

Stability studies were carried out at 40° C./75%RH and 60° C. for two month and found that the tablet composition was stable.

The tablets of example 2 and 3 were manufactured using the procedure comprising the following steps: valsartan and mannitol was done in a rapid mixer granulator and mixed for 5 to 20 minutes. Binder solution (povidone K-29/32 in purified water) is added to contents of rapid mixer granulator and mixed to get dough mass. The dough mass was dried, and dried granules passed through #18.

The blending of ingredients including above dried granules, maize starch and/or lactose or microcrystalline cellulose or dibasic calcium phosphate, croscarmellose sodium and magnesium stearate were weighted, transferred into a blender, and mixed to form a homogeneous power mixture; the resultant mixture was compressed into tablets of appropriate weight and hardness to obtain a valsartan tablet; and the tablets was coated with opadry.

### Example 2

### Preparation of Valsartan Tablet:

The tablets were prepared using the materials listed in table.

| Component | Weight (mg)/ Tablet | | | | % (w/w) |
|---|---|---|---|---|---|
| Valsartan | 40 | 80 | 160 | 320 | 32 |
| Mannitol | 72.25 | 144.5 | 289 | 578 | 57.8 |
| Povidone | 6.25 | 12.5 | 25 | 50 | 5.0 |
| Purified water | q.s | q.s | q.s | q.s | — |
| Microcrystalline | 2.5 | 5 | 10 | 20 | 2 |
| cellulose | | | | | |
| Croscarmellose sodium | 2.5 | 5 | 10 | 20 | 2 |
| Calcium stearate | 1.5 | 3 | 6 | 12 | 1.2 |
| Tablet weight | 125 | 250 | 500 | 1000 | — |
| Opadry | 2.5 | 5 | 10 | 20 | 2 |

### Example 3

### Preparation of Valsartan Tablet:

The tablets were prepared using the materials listed in table.

| Component | Weight (mg)/ Tablet | | | | % (w/w) |
|---|---|---|---|---|---|
| Valsartan | 40 | 80 | 160 | 320 | 32 |
| Mannitol | 66 | 132 | 264 | 528 | 52.8 |
| Povidone | 10 | 20 | 40 | 80 | 8.0 |
| Purified water | q.s | q.s | q.s | q.s | — |
| Lactose | 2.5 | 5 | 10 | 20 | 2 |
| Dibasic calcium | 2.5 | 5 | 10 | 20 | 2 |
| phosphate | | | | | |
| Croscarmellose | 2.5 | 5 | 10 | 20 | 2 |
| sodium | | | | | |
| Magnesium stearate | 1.5 | 3 | 6 | 12 | 1.2 |
| Tablet weight | 125 | 250 | 500 | 1000 | — |
| Opadry | 2.5 | 5 | 10 | 20 | 2 |

The tablets of example 4, 5, 6 and 7 were manufactured using the procedure comprising the following steps: valsartan, hydrochlorothiazide and mannitol was done in a rapid mixer granulator and mixed for 5 to 20 minutes. Binder solution (povidone K-29/32 in purified water) is added to contents of rapid mixer granulator and mixed to get dough mass. The dough mass was dried, and dried granules passed through #18.

The blending of ingredients including above dried granules, maize starch or microcrystalline cellulose, croscarmellose sodium and magnesium stearate were weighted, transferred into a blender, and mixed to form a homogeneous power mixture; the resultant mixture was compressed into tablets of appropriate weight and hardness to obtain a valsartan and hydrochlorothiazide tablet; and the tablets was coated with opadry.

### Example 4

### Preparation of Valsartan and Hydrochlorothiazide Tablet:

The tablets were prepared using the materials listed in table.

| Component | Weight (mg)/ Tablet | | % (w/w) |
|---|---|---|---|
| Valsartan | 80 | 160 | 32 |
| Hydrochlorothiazide | 12.5 | 25 | 5 |
| Mannitol | 142 | 284 | 56.8 |
| Povidone | 2.5 | 5 | 1 |
| Purified water | q.s | q.s | — |
| Maize starch | 5 | 10 | 2 |
| Croscarmellose sodium | 5 | 10 | 2 |
| Magnesium stearate | 3 | 6 | 1.2 |
| Tablet weight | 250 | 500 | — |
| Opadry | 5 | 10 | 2 |

### Example 5

### Preparation of Valsartan and Hydrochlorothiazide Tablet:

The tablets were prepared using the materials listed in table.

| Component | Weight (mg)/ Tablet | | % (w/w) |
|---|---|---|---|
| Valsartan | 80 | 160 | 32 |
| Hydrochlorothiazide | 12.5 | 25 | 5 |
| Mannitol | 132 | 264 | 52.8 |
| Povidone | 12.5 | 25 | 5 |
| Purified water | q.s | q.s | — |
| Microcrystalline cellulose | 5 | 10 | 2 |
| Croscarmellose sodium | 5 | 10 | 2 |
| Magnesium stearate | 3 | 6 | 1.2 |
| Tablet weight | 250 | 500 | — |
| Opadry | 5 | 10 | 2 |

### Example 6

### Preparation of Valsartan and Hydrochlorothiazide Tablet:

The tablets were prepared using the materials listed in table.

| Component | Weight (mg)/ Tablet | | % (w/w) |
|---|---|---|---|
| Valsartan | 160 | 320 | 32 |
| Hydrochlorothiazide | 12.5 | 25 | 2.5 |
| Mannitol | 296.5 | 593 | 59.3 |
| Povidone | 5 | 10 | 1 |
| Purified water | q.s | q.s | — |
| Maize starch | 10 | 20 | 2 |
| Croscarmellose sodium | 10 | 20 | 2 |
| Magnesium stearate | 6 | 12 | 1.2 |
| Tablet weight | 500 | 1000 | — |
| Opadry | 10 | 20 | 2 |

### Example 7

### Preparation of Valsartan and Hydrochlorothiazide Tablet:

The tablets were prepared using the materials listed in table.

| Component | Weight (mg)/ Tablet | % (w/w) |
|---|---|---|
| Valsartan | 320 | 32 |
| Hydrochlorothiazide | 12.5 | 1.25 |
| Mannitol | 605.5 | 60.55 |
| Povidone | 10 | 1 |
| Purified water | q.s | — |
| Maize starch | 20 | 2 |
| Croscarmellose sodium | 20 | 2 |
| Magnesium stearate | 12 | 1.2 |
| Tablet weight | 1000 | — |
| Opadry | 20 | 2 |

The tablets of example 8, 9 and 10 were manufactured using the procedure comprising the following steps: valsartan, amlodipine and mannitol was done in a rapid mixer granulator and mixed for 5 to 20 minutes. Binder solution (povidone K-29/32 in purified water) is added to contents of rapid mixer granulator and mixed to get dough mass. The dough mass was dried, and dried granules passed through #18.

The blending of ingredients including above dried granules, maize starch, croscarmellose sodium and magnesium stearate were weighted, transferred into a blender, and mixed to form a homogeneous power mixture; the resultant mixture was compressed into tablets of appropriate weight and hardness to obtain a valsartan and amlodipine tablet; and the tablets was coated with opadry.

### Example 8

### Preparation of Valsartan and Amlodipine Tablet:

The tablets were prepared using the materials listed in table.

| Component | Weight (mg)/ Tablet | | % (w/w) |
|---|---|---|---|
| Valsartan | 160 | 320 | 32 |
| Amlodipine besylate | 6.94 | 13.88 | 1.4 |
| Eq to amlodipine | | | |
| Mannitol | 302.06 | 604.12 | 60.4 |
| Povidone | 5 | 10 | 1 |
| Purified water | q.s | q.s | — |
| Maize starch | 10 | 20 | 2 |
| Croscarmellose sodium | 10 | 20 | 2 |
| Magnesium stearate | 6 | 12 | 1.2 |
| Tablet weight | 500 | 1000 | — |
| Opadry | 10 | 20 | 2 |

### Example 9

### Preparation of Valsartan and Amlodipine Tablet:

The tablets were prepared using the materials listed in table.

| Component | Weight (mg)/ Tablet | % (w/w) |
|---|---|---|
| Valsartan | 320 | 32 |
| Amlodipine besylate | 6.94 | 0.7 |
| Eq to amlodipine | | |
| Mannitol | 611.06 | 61.1 |
| Povidone | 10 | 1 |
| Purified water | q.s | — |
| Maize starch | 20 | 2 |
| Croscarmellose sodium | 20 | 2 |
| Magnesium stearate | 12 | 1.2 |
| Tablet weight | 1000 | — |
| Opadry | 20 | 2 |

### Example 10

### Preparation of Valsartan and Amlodipine Tablet:

The tablets were prepared using the materials listed in table.

| Component | Weight (mg)/ Tablet | % (w/w) |
|---|---|---|
| Valsartan | 160 | 32 |
| Amlodipine besylate | 13.88 | 2.8 |
| Eq to amlodipine | | |
| Mannitol | 295.12 | 59.0 |
| Povidone | 5 | 1 |
| Purified water | q.s | — |
| Maize starch | 10 | 2 |
| Croscarmellose sodium | 10 | 2 |
| Magnesium stearate | 6 | 1.2 |
| Tablet weight | 500 | — |
| Opadry | 10 | 2 |

The tablets of example 11, 12, 13 and 14 were manufactured using the procedure comprising the following steps: valsartan, hydrochlorothiazide, amlodipine and mannitol was done in a rapid mixer granulator and mixed for 5 to 20 minutes. Binder solution (povidone K-29/32 in purified water) is added to contents of rapid mixer granulator and mixed to get dough mass. The dough mass was dried, and dried granules passed through #20.

The blending of ingredients including above dried granules, maize starch, croscarmellose sodium and magnesium stearate were weighted, transferred into a blender, and mixed to form a homogeneous power mixture; the resultant mixture was compressed into tablets of appropriate weight and hardness to obtain a valsartan, hydrochlorothiazide and amlodipine tablet; and the tablets was coated with opadry.

### Example 11

### Preparation of Valsartan, Hydrochlorothiazide and Amlodipine Tablet:

The tablets were prepared using the materials listed in table.

| Component | Weight (mg)/ Tablet | | % (w/w) |
|---|---|---|---|
| Valsartan | 160 | 320 | 32 |
| Hydrochlorothiazide | 12.5 | 25 | 2.5 |
| Amlodipine besylate | 6.94 | 13.88 | 1.4 |
| Eq to amlodipine | | | |
| Mannitol | 289.56 | 579.12 | 57.9 |
| Povidone | 5 | 10 | 1 |
| Purified water | q.s | q.s | — |
| Maize starch | 10 | 20 | 2 |
| Croscarmellose sodium | 10 | 20 | 2 |
| Magnesium stearate | 6 | 12 | 1.2 |
| Tablet weight | 500 | 1000 | — |
| Opadry | 10 | 20 | 2 |

### Example 12

### Preparation of Valsartan, Hydrochlorothiazide and Amlodipine Tablet:

The tablets were prepared using the materials listed in table.

| Component | Weight (mg)/ Tablet | % (w/w) |
|---|---|---|
| Valsartan | 160 | 32 |
| Hydrochlorothiazide | 12.5 | 2.5 |
| Amlodipine besylate | 13.88 | 2.8 |
| Eq to amlodipine | | |
| Mannitol | 282.62 | 56.5 |
| Povidone | 5 | 1 |
| Purified water | q.s | — |
| Maize starch | 10 | 2 |
| Croscarmellose sodium | 10 | 2 |
| Magnesium stearate | 6 | 1.2 |
| Tablet weight | 500 | — |
| Opadry | 10 | 2 |

### Example 13

### Preparation of Valsartan, Hydrochlorothiazide and Amlodipine Tablet:

The tablets were prepared using the materials listed in table.

| Component | Weight (mg)/ Tablet | % (w/w) |
|---|---|---|
| Valsartan | 160 | 32 |
| Hydrochlorothiazide | 25 | 5 |
| Amlodipine besylate | 13.88 | 2.8 |
| Eq to amlodipine | | |
| Mannitol | 270.12 | 54.0 |
| Povidone | 5 | 1 |
| Purified water | q.s | — |
| Maize starch | 10 | 2 |
| Croscarmellose sodium | 10 | 2 |
| Magnesium stearate | 6 | 1.2 |
| Tablet weight | 500 | — |
| Opadry | 10 | 2 |

### Example 14

### Preparation of Valsartan, Hydrochlorothiazide and Amlodipine Tablet:

The tablets were prepared using the materials listed in table.

| Component | Weight (mg)/ Tablet | % (w/w) |
|---|---|---|
| Valsartan | 160 | 32 |
| Hydrochlorothiazide | 25 | 5 |
| Amlodipine besylate | 6.94 | 1.4 |
| Eq to amlodipine | | |
| Mannitol | 277.06 | 55.4 |
| Povidone | 5 | 1 |
| Purified water | q.s | — |
| Maize starch | 10 | 2 |
| Croscarmellose sodium | 10 | 2 |
| Magnesium stearate | 6 | 1.2 |
| Tablet weight | 500 | — |
| Opadry | 10 | 2 |

### Example 15

### Preparation of Valsartan and Amlodipine Tablet:

The tablets were prepared using the materials listed in table.

| Component | Weight (mg)/ Tablet | % (w/w) |
|---|---|---|
| Valsartan layer | | |
| Valsartan | 160 | 32 |
| Mannitol | 309 | 61.8 |
| Povidone | 5 | 1 |
| Purified water | q.s | — |
| Maize starch | 10 | 2 |
| Croscarmellose sodium | 10 | 2 |
| Magnesium stearate | 6 | 1.2 |
| Valsartan layer weight | 500 | — |
| Amlodipine layer | | |
| Amlodipine besylate | 13.88 | 6.94 |
| Eq to amlodipine | | |
| Microcrystalline cellulose | 122.12 | 61.06 |
| Dibasic calcium | 60 | 30 |
| phosphate | | |
| Magnesium sterate | 4 | 2 |
| Amlodipine layer | 200 | — |
| weight | | |
| Tablet weight | 700 | — |
| Opadry | 14 | 2 |

1. A pharmaceutical composition comprising valsartan or a pharmaceutically acceptable salt thereof with mannitol as a filler and povidone as binder, and optionally one or more additional excipients.

2. A pharmaceutical composition comprising valsartan or a pharmaceutically acceptable salt thereof with mannitol as a filler, povidone as binder in combination with hydrochlorothiazide or amlodipine or both; and optionally one or more additional excipients.

3. (canceled)

4. (canceled)

5. The composition as claimed in claim 1, wherein the ratio of valsartan or a pharmaceutically acceptable salt thereof to mannitol is about 1:0.25 to about 1:5.

6. The composition as claimed in claim 1, wherein the ratio of valsartan or a pharmaceutically acceptable salt thereof to povidone 1:0.01 to about 1:0.5.

7. The composition as claimed in claim 1, wherein the ratio of povidone to mannitol is about 1:30 to about 1:90.

8. (canceled)

9. The composition as claimed in claim 1, wherein the valsartan or a pharmaceutically acceptable salt thereof is in the concentration of about 20 to about 34%, based on total weight of pharmaceutical composition.

10. The composition as claimed in claim 1 or 2, wherein the additional excipient is selected from pharmaceutical filler, disintegrants, binders, lubricants, glidants, coating agents and coloring agents and a mixture thereof.

11. The composition as claimed in claim 10, wherein the filler is selected from mannitol, microcrystalline cellulose, calcium carbonate, dibasic calcium phosphate, lactose, magnesium carbonate, magnesium oxide, lactose anhydrous, isomalt, sorbitol or mixtures thereof.

12. (canceled)

13. The composition as claimed in claim 10, wherein the lubricants is selected from magnesium stearate, calcium stearate, stearic acid, a salt of stearic acid, talc, sodium stearyl fumarate, glyceryl behenate, magnesium silicate, magnesium trisilicate, macrogol, talc, hydrogenated castor oil or mixtures thereof.

14. (canceled)

15. The composition as claimed in claim 10, wherein the disintegrant is selected from croscarmellose sodium, starch, sodium starch glycolate, crospovidone, carboxymethyl cellulose calcium, carboxymethylcellulose sodium, magnesium aluminium silicate or mixtures thereof.

16. (canceled)

17. (canceled)

18. The composition as claimed in claim 10, wherein the binder is selected from polyvinylpyrrolidone k-30, hydroxypropyl cellulose, hydroxypropyl methyl cellulose, hydroxypropyl cellulose (low-substituted), starch or mixtures thereof.

19. The composition as claimed in claim 18, wherein the binder is polyvinylpyrrolidone k-30 and/or hydroxypropyl cellulose.

20. (canceled)

21. (canceled)

22. A process for preparation of pharmaceutical composition which comprises mixing valsartan or a pharmaceutically acceptable salt thereof, mannitol and povidone, and optionally one or more additional excipients.

23. A process for preparation of pharmaceutical composition as claimed in claim 22, comprises mixing valsartan or a pharmaceutically acceptable salt thereof in combination with hydrochlorothiazide or amlodipine or both; mannitol and povidone, and optionally one or more additional excipients.

24. (canceled)

25. (canceled)

26. The process as claimed in claim 22, wherein the tablets are prepared by direct compression, wet granulation and slugging method or roll compaction.

27. The process as claimed in claim 22, wherein the tablets are prepared by wet granulation.

28. (canceled)

29. (canceled)

30. (canceled)

31. (canceled)

32. (canceled)
